(19) United States
(12) Patent Application Publication
(10) Pub. No.: US 2022/0331568 A1
(43) Pub. Date: Oct. 20, 2022

(54) NEOVAGINAL STENT SYSTEM

(71) Applicant: MEDSTAR HEALTH, INC., Columbia, MD (US)

(72) Inventors: Gabriel Del Corral, Baltimore, MD (US); Colleen Lindo, Baltimore, MD (US); Andrew Hodge, Baltimore, MD (US); Angela Jaimes, Baltimore, MD (US)

(21) Appl. No.: 17/715,324

(22) Filed: Apr. 7, 2022

Related U.S. Application Data
(60) Provisional application No. 63/176,379, filed on Apr. 19, 2021.

(51) Int. Cl. A61M 29/00; A61B 17/42

(52) U.S. Cl. CPC A61M 29/00; A61B 17/42; A61M 2210/1475

(57) ABSTRACT

A neovaginal stent system including a neovaginal stent configured to dilate and maintain the patency of a neovagina as well as irrigate the neovagina. The neovaginal stent includes a sponge casing, a balloon within an inner cavity of the sponge casing and a membrane surrounding the sponge casing.

### CROSS-REFERENCE TO RELATED APPLICATIONS

The present application claims priority to U.S. Provisional Application No. 63/176,379 entitled “Neovaginal Stent System” filed on Apr. 19, 2021 and incorporated by reference herein in its entirety.

### TECHNICAL FIELD

The present disclosure relates to a neovaginal stent system.

### BACKGROUND

Transgender vaginoplasty has become more popular among patients seeking genital reconstruction. The functional success of the surgery is directly correlated with the patency and length of the vaginal canal, which is accomplished with a vaginal dilator. Such a vaginal dilator is a device that is shaped and configured to maintain the vaginal pocket after a vaginoplasty procedure and/or a device shaped and configured to create the vaginal pocket during a vaginoplasty procedure. Traditional vaginal dilation after gender affirmation surgery is a painful, tedious, time consuming, year-long process that a patient must endure to ensure surgical success. Failure to perform vaginal dilation can lead to vaginal stenosis, patient regret, and mental suffering. Traditionally, vaginal dilation has been done with hard dilators, which are devices that are shaped and configured to maintain the vaginal pocket after a vaginoplasty procedure. Use of such dilators requires steady pressure against the newly-created vaginal pocket (neovagina), which can potentially injure the surrounding structures, such as the rectum.

### SUMMARY

The present disclosure relates to neovaginal devices, systems and kits. In an aspect, a neovaginal stent system comprises a neovaginal stent. Such a stent includes a sponge casing having an exterior surface, an interior surface, and a sponge body therebetween. The interior surface defines an inner cavity. The stent further includes a balloon disposed within the inner cavity of the sponge casing and having an inflation lumen in fluid communication with an inflation port. The stent further comprises a permeable or semi-permeable membrane disposed on the exterior surface of the sponge casing. The neovaginal stent is sized and configured to dilate and maintain the patency of a neovagina.

In certain aspects, the present disclosure provides a kit comprising a plurality of differently sized neovaginal stents. Each neovaginal stent comprises a sponge casing having an exterior surface, an interior surface, and a sponge body therebetween. The interior surface defines an inner cavity. A balloon is disposed within the inner cavity of the sponge casing and has an inflation lumen in fluid communication with an inflation port. A permeable or semi-permeable membrane is disposed on the exterior surface of the sponge casing. Each of the plurality of neovaginal stents is sized and configured to dilate and maintain the patency of a neovagina.

### BRIEF DESCRIPTION OF DRAWINGS

FIG. 1 is proximal-distal cross-sectional view of a neovaginal stent system according to an aspect of the present disclosure.

FIG. 2 is a mediolateral cross-sectional view of a neovaginal stent system according to an aspect of the present disclosure.

### DETAILED DESCRIPTION

As used herein with respect to a described element, the terms “a,” “an,” and “the” include at least one or more of the described element(s) including combinations thereof unless otherwise indicated. Further, the terms “or” and “and” refer to “and/or” and combinations thereof unless otherwise indicated. By “substantially” is meant that the shape or configuration of the referenced element need not have the mathematically exact described shape or configuration of the referenced element but can have a shape or configuration that is recognizable by one skilled in the art as generally or approximately having the described shape or configuration of the referenced element. As such “substantially” refers to the complete or nearly complete extent of a referenced characteristic. The exact allowable degree of deviation from the characteristic will be so as to have the same overall result as if the absolute characteristic were obtained. The terms “inner,” and “outer,” refer to the position of elements as they are depicted in the drawings. The terms “first,” “second,” etc. are used to distinguish one element from another and not used in a quantitative sense unless indicated otherwise. Thus, a “first” element described below could also be termed a “second” element. By “integral” or “integrated” is meant that the referenced components are fabricated as one piece or multiple pieces affixed during manufacturing such that the components are a unitary and continuous piece after manufacturing. Such referenced components are not separable without damaging the integrity (i.e. tearing) of either of the components as opposed to two-piece or multi-piece components that are assembled together after manufacturing, such as prior to or during surgical insertion, and where the components can be separated. As used herein a “patient” includes a mammal such as a human being. All device, assemblies, and kits as described herein are used for medical purposes and are therefore sterile.

The present disclosure relates to a neovaginal stent system that is used to maintain the patency of a neovagina and to provide irrigation of the vagina after a vaginoplasty procedure. Such a neovaginal stent system can provide many benefits including the ability for the patient to use the stent system not only in the immediate post-operative phase but also during the months after surgery. Such a system can provide a progressive, soft expansion of the neovaginal canal that can be tolerated by the patient. Additionally, such a system can be used for various types of vaginoplasties including but not limited to intestinal and peritoneal vaginoplasties.

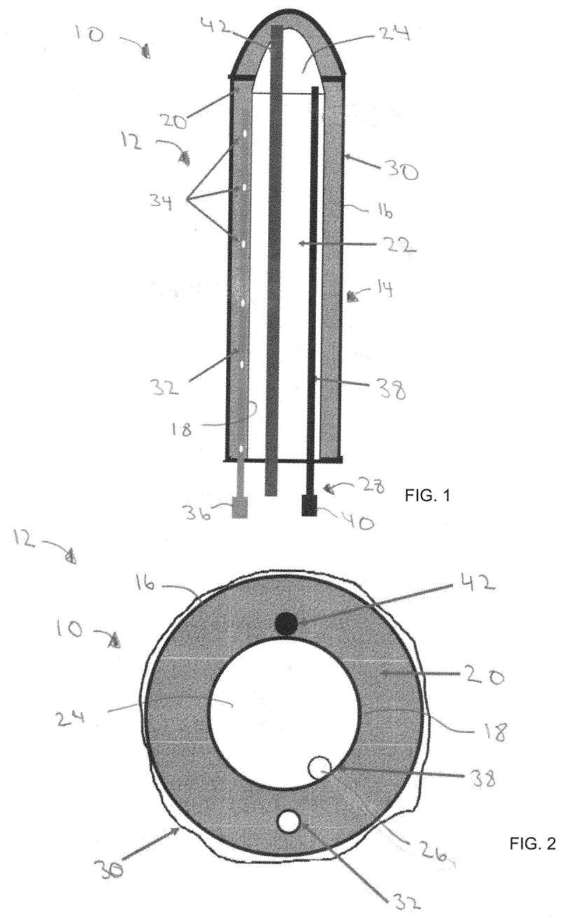

Referring to FIGS. 1 and 2, a neovaginal stent system 10 comprises a neovaginal stent 12. Neovaginal stent 12 includes a sponge casing 14 having an outer surface 16, an inner surface 18 and a sponge body 20 therebetween. Inner surface 18 defines an interior cavity 22. A balloon 24 is disposed within interior cavity 22 of sponge casing 14 and has an inflation lumen 26 in fluid communication with an inflation port 28. Although only one elongated balloon is depicted the balloon can comprise a plurality of balloons that have, for example, spherical shapes encased in an elongated sheath. Neovaginal stent 12 further comprises a permeable or semi-permeable membrane 30 disposed on outer surface 16 of sponge casing 14. The membrane can encase the sponge casing. Neovaginal stent 12 is sized and configured to dilate and maintain the patency of a neovaginal canal.

Neovaginal stent system 10 can further include a drainage catheter 32 disposed within sponge body 20 and having drainage apertures 34 disposed along the length of the drainage catheter. The neovaginal stent system can further include a drainage/collection bag (not shown), with which the drainage catheter is in fluid communication. The drainage catheter can connect to the drainage bag via a luer lock 36, for example, or another type of connection device. Alternatively, the drainage catheter can be in fluid communication with a suction source such as a suction bulb or other aspiration device. The neovaginal stent system 10 can also include an inflation catheter 38 disposed within interior cavity 22 of sponge casing 14. An inflation catheter 38 can define the inflation lumen and the inflation port of the balloon. The inflation port can be in fluid communication with an inflation device, such as a syringe (not shown), for example. In embodiments including an inflation catheter, the inflation catheter can connect to the syringe via a luer lock 40 or other type of connection device. Neovaginal stent system 10 can further include a stylet 42 to assist in inserting, positioning, and manipulating neovaginal stent 12. Such a stylet can be permanently or transiently attached to the neovaginal stent.

The sponge casing as well as the drainage catheter can collect fluid, such as blood and mucus, (e.g. in the case of intestinal vaginoplasty) during the initial post-operative phase as well as after the initial post-operative phase such as during post-operative days two and four, for example, before removing the neovaginal stent system. Such drainage or aspiration can ensure that blood and other fluids do not remain stagnant in the vaginal canal, which can interfere with the formation of the vaginal graft. Later in recovery, vaginal douching can be performed using the neovaginal stent system. In this case, the sponge casing and the drainage catheter serve to remove the saline recently infiltrated for douching. Such vaginal douching is usually done twice a day for the first 3-6 months but the time frame can vary. The neovaginal stent of the neovaginal stent system can also provide structure and patency to dilate the neovagina. The outer surface of the sponge casing or membrane can include ruler markings to provide an indication of the length/depth and width of the inflated balloon. Further, the width of the balloon could also be determined as correlative of balloon pressure. For example, an inflated balloon having a width of 2 centimeters could indicate a balloon pressure of five psi; an inflated balloon having a width of three centimeters could indicate a balloon pressure of 10 psi, etc.

The present disclosure further includes kits comprising neovaginal stents having various different sizes to accommodate different neovagina sizes.

The neovaginal stent system can remain in place for a number of days after surgery, such as, for example, five days after surgery. The neovaginal stent can be used for the first year after surgery for example. As such, the stent system can replace standard plastic vaginal dilators, which patients typically have to use three times a day for the first six months and then twice to once a day for the following six months. The membrane can serve as a barrier between the sponge casing and the vaginal tissue such that the tissue graft is not in direct contact with the sponge casing to prevent or mitigate the sponge casing from sucking in the tissue graft. As such, the membrane allows fluid to be infiltrated and absorbed by the sponge casing without affecting the adjacent structures such as the tissue graft, the bowel, etc. Exemplary membrane materials include non-degradable polymers including polyethylene terephthalate (PET), nylon 6,6, polyurethane (PU), polytetrafluoroethylene (PTFE), polyethylene (PE), polysiloxanes and poly(methylmethacrylate) (PMMA), modified polyacrylonitrile (PAN) [14] and polyether imide (PEI). The membrane can also be fabricated from a bioresorbable polymer that is designed to degrade within the body and be absorbed naturally when its function has been accomplished These degradation characteristics differ from polymer to polymer, and can vary from swelling to dissolution by hydrolysis, for instance, when being exposed to body fluids. Bioresorbable materials degrade products that are normal metabolites of the body. Some examples of degradable polymers are polylactide, polyglycolide, polycaprolactone and polyhyaluronic acid esters, but also natural polymers like collagen, chitosan, for example. The membrane could also be similar to a dialysis membrane, such as synthetic membrane. Non-limiting examples of membrane materials includes hydrophilic or hydrophilized copolymers (e.g. polyethylene vinyl alcohol, polymethyl methacrylate or modified polyacrylonitrile); hydrophilic blends that are mostly prepared by mixing high glass transition temperature (Tg) hydrophobic polymers (e.g. polysulfone (PSf) or polyarylether sulfone (PES, PAES)) with hydrophilic polymers (polyvinyl pyrrolidone (PVP) or aliphatic/aromatic polyamides). In certain aspects, the membrane is a polymer-based membrane that may allow blood cells to pass through. A blood cell is typically 5-8 um in diameter so the polymer-based membrane can allow materials 10 um wide to pass. However, the membrane could also only allow water and much smaller materials to pass through.

A non-limiting example of a method of using a neovaginal stent system after a vaginoplasty will now be described. The patient inserts the neovaginal stent system into the neovagina and inflates the balloon for twenty to thirty minutes. Prior to removing the neovaginal stent system, the patient irrigates the neovagina with saline. The drainage catheter in conjunction with an aspiration device can be used to suction blood, other fluid(s), and graft particles so that such substances are removed and do not remain stagnant in the neovaginal canal. The patient then deflates the balloon and removes the device from the neovagina.

Although the drawings show certain elements of a neovaginal stent, it should be noted that such elements can be included in other embodiments or aspects illustrated in other drawings or otherwise described in the specification. In other words, each of the disclosed aspects and embodiments of the present disclosure may be considered individually or in combination with other aspects and embodiments of the disclosure including patent applications incorporated by reference herein.

1. A neovaginal stent system comprising:
a neovaginal stent comprising:
a sponge casing having an exterior surface, an interior surface, and a sponge body therebetween, the interior surface defining an inner cavity;
a balloon disposed within the inner cavity of the sponge casing and having an inflation lumen in fluid communication with an inflation port; and
a permeable or semi-permeable membrane disposed on the exterior surface of the sponge casing, wherein the neovaginal stent is sized and configured to dilate and maintain the patency of a neovagina.

2. The neovaginal stent system of claim 1, further comprising a drainage catheter disposed within the sponge body, the drainage catheter having apertures disposed along the length of the drainage catheter.

3. The neovaginal stent system of claim 2, further comprising a drainage bag, the drainage catheter being in fluid communication with the drainage bag.

4. The neovaginal stent system of claim 2, further comprising an aspiration device, the drainage catheter being in fluid communication with the aspiration device.

5. The neovaginal stent system of claim 1, further comprising an inflation catheter disposed within the interior cavity of the sponge casing and defining the inflation lumen and the inflation port.

6. The neovaginal stent system of claim 1, wherein the inflation port is in fluid communication with an inflation device.

7. The neovaginal stent system of claim 6, wherein the inflation device is a syringe.

8. The neovaginal stent system of claim 1, further comprising a stylet disposed within the sponge body.

9. A kit comprising:
a plurality of differently sized neovaginal stents, each neovaginal stent comprising:
a sponge casing having an exterior surface, an interior surface, and a sponge body therebetween, the interior surface defining an inner cavity;
a balloon disposed within the inner cavity of the sponge casing and having an inflation lumen in fluid communication with an inflation port; and
a permeable or semi-permeable membrane disposed on the exterior surface of the sponge casing, wherein each of the plurality of differently sized neovaginal stents is sized and configured to dilate and maintain the patency of a neovagina.
